(19) United States
(12) Patent Application Publication
(10) Pub. No.: US 2024/0335428 A1
(43) Pub. Date: Oct. 10, 2024

(54) PATEAMINE A DERIVATIVE FOR TREATING PANCREATIC CANCER

(71) Applicants: Baylor University, Waco, TX (US); The Trustees of Columbia University in the City of New York, New York, NY (US)

(72) Inventors: Daniel Romo, Waco, TX (US); Kenneth G. Hull, Waco, TX (US); Haoran Xue, Waco, TX (US); Mingzhao Zhu, Waco, TX (US); Susan E. Bates, New York, NY (US); Maryam Safari, New York, NY (US); Antonio Fojo, New York, NY (US)

(73) Assignees: Baylor University, Waco, TX (US); The Trustees of Columbia University in the City of New York, New York, NY (US)

(21) Appl. No.: 18/627,405

(22) Filed: Apr. 4, 2024

Related U.S. Application Data
(60) Provisional application No. 63/495,015, filed on Apr. 7, 2023.

(51) Int. Cl. A61K 31/429; A61K 9/00; A61P 35/00

(52) U.S. Cl. CPC A61K 31/429; A61K 9/0019; A61P 35/00

(57) ABSTRACT

Method for treating pancreatic cancer using des-methyl pateamine A (DMPatA) or a pharmaceutically acceptable salt thereof, or des-methyl des-amino pateamine A (DMDAPatA) or a pharmaceutically acceptable salt thereof.

### CROSS-REFERENCE TO RELATED APPLICATION

This application claims the benefit of Provisional Application No. 63/495,015, filed Apr. 7, 2023, which is expressly incorporated herein by reference in its entirety.

### BACKGROUND

Elevated protein synthesis is a vital feature of many malignant phenotypes and often arises as a result of increased oncogenic signaling flux channeled to eukaryotic initiation factor 4F (eIF4F) to promote cap-dependent mRNA translation. Eukaryotic initiation factor 4A (eIF4A), an RNA helicase enzyme, is part of the cellular eIF4F translation initiation complex which has been described as a key element of the cap-dependent translational initiation process. Thus, selective inhibition of eIF4A using synthetic drug candidates is a promising avenue to explore for targeted cancer therapy.

eIF4A is an essential component of the eukaryotic initiation factor 4F (eIF4F) complex with RNA helicase activity that unwinds RNA and presents it to the 43S ribosome subunit during translation initiation.

The marine natural product pateamine A which was isolated from the marine sponge Mycale sp is an antiproliferative agent that binds to eIF4A. Des-methyl des-amino pateamine A (DMDAPatA) is the first lead compound from the series and is more stable and easier to synthesize than the parent natural product PatA.

Pancreatic ductal adenocarcinoma (PDAC) is one of the most fatal forms of cancer due to the lack of early diagnosis and limited response to known treatments.

PDAC resistance to therapy is thought to comprise both cellular (e.g., activating KRAS mutations found in 90% of pancreatic cancers, elevated C-MYC expression with high protein levels in over 40% of cases) and microenvironment components (e.g., dense extra cellular matrix with elevated hyaluronic acid levels and elevated expression of CD44).

A need exists for therapeutic agents for the treatment of pancreatic ductal adenocarcinoma (PDAC). The present disclosure seeks to fulfill this need and provides further related advantages.

### BRIEF DESCRIPTION OF THE FIGURES

The foregoing aspects and many of the attendant advantages of this invention will become more readily appreciated as the same become better understood by reference to the following detailed description, when taken in conjunction with the accompanying drawings.

FIG. 1A compares tumor size (mm³) growth over time (28 days) for the vehicle. The numbers in the key represent the individual mice in the experiment. Each number is one mouse. Mean symbol represents the mean drug effect on all mice combined.

FIG. 1B compares tumor size (mm³) growth over time (28 days) for a dose of 0.1 mg/kg DMPatA (MZ735). The numbers in the key represent the individual mice in the experiment. Each number is one mouse. Mean symbol represents the mean drug effect on all mice combined.

FIG. 1C compares tumor size (mm³) growth over time (28 days) for a dose of 0.075 mg/kg DMPatA (MZ735). The numbers in the key represent the individual mice in the experiment. Each number is one mouse. Mean symbol represents the mean drug effect on all mice combined.

FIGS. 2A-2D are viability curves of DMDAPatA and DMPatA in four representative PDAC cell lines: MIA PaCa-2; Panc-1; Capan-1; and AsPC-1. PDAC cell lines were treated with DMPatA and DMDAPatA at the indicated concentrations for 48 h and 72 h. Cell viability was determined using Cell-Titer Glo (Promega). Viability (%, Y-axis) was plotted against drug concentration (log nM, X-axis). In the figures, MZ is DMPatA and PatA is DMDAPatA.

FIGS. 3A and 3B are images of western blots showing the effect of DMDAPatA and DMPatA in PDAC cell lines (MiaPaca-2 and AsPc-1). In the figures, MZ735 is DMPatA and PatA is DMDAPatA.

FIG. 4 compares images of western blots illustrating overexpression of MYC under a powerful promoter. In the figure, MZ is DMPatA.

FIGS. 5A and 5B are images of western blots showing the effect of DMPatA and DMDAPatA on oncoproteins c-MYC and Cyclin D1 in PDAC cell lines: MIA PaCa-2; PANC-1; Capan-1; and AsPC-1. In the figures, MZ is DMPatA and PatA is DMDAPatA.

FIGS. 6A and 6B illustrate that representative eIF4A inhibitors (DMPatA and DMDAPatA) reduce the levels of hyaluronic acid and CD44, which play important roles in the microenvironment. CD44 is the cell surface receptor for hyaluronic acid. Dense stroma with high levels of hyaluronic acid and extracellular matrix are among factors that are said to contribute to drug resistance in PDAC due to reduced permeability of anticancer therapies. Reduction of CD44 could facilitate the activity of chemotherapy or other combination therapy. MIA PaCa-2 cells were treated with 5 nM of DMPatA for 24 h versus untreated control. Cells were stained for HA using biotinylated-HABP and anti-CD44. FIG. 6A compares the analysis of the corrected total cell fluorescence (CTCF) based on data extracted from confocal imaging (P<0.05) and FIG. 6B compares western blot showing the effect of DMPatA and DMDAPatA on stromal proteins HAS3 and CD44. In the figures, MZ and MZ735 are DMPatA and PatA is DMDAPatA.

FIGS. 7A and 7B illustrate proteomics and RNA-seq analysis identifying key regulatory molecules predicted to be responsible for the observed changes in the expression patterns following treatment with eIF4A inhibitors. FIG. 7A illustrates the IPA Upstream Regulator analysis used to identify key regulatory molecules predicted to be responsible for the differences observed in protein expression patterns following DMPatA treatment from a proteomics dataset. Proteins with Z-score>1 were considered to be activated (light gray bars) and those with Z-scores of <−1 were considered to be inhibited (dark gray bars). Heat map is showing only proteins with z-score cut off of ≤−1.8 for a total of 291 predicted upstream regulatory proteins. FIG. 7B illustrates the Virtual Inference of Protein-activity by Enriched Regulon analysis (VIPER) algorithm analysis used to identify differentially regulated master regulators, by examining data extracted from RNA-seq datasets of samples treated with DMPatA compared to untreated control. In the figures, MZ and MZ735 are DMPatA.

FIG. 8 is a schematic illustration of a de novo synthesis of DMPatA.

FIGS. 9A-9D are schematic illustrations of the syntheses of Fragments D, C, F, and G, respectively.

### SUMMARY

In one aspect, the present disclosure provides a method for treating pancreatic cancer. In the method, a therapeutically effective amount of des-methyl pateamine A (DMPatA) or a pharmaceutically acceptable salt thereof, or des-methyl des-amino pateamine A (DMDAPatA) or a pharmaceutically acceptable salt thereof, is administered to a subject in need thereof.

In certain embodiments of the method, pancreatic cancer is pancreatic ductal adenocarcinoma.

In certain embodiments of the method, des-methyl pateamine A (DMPatA) or a pharmaceutically acceptable salt thereof, or des-methyl des-amino pateamine A (DMDAPatA) or a pharmaceutically acceptable salt thereof, is administered by intravenous injection. In other embodiments, DMPatA or a pharmaceutically acceptable salt thereof, or des-methyl des-amino pateamine A (DMDAPatA) or a pharmaceutically acceptable salt thereof, is administered intraperitoneally. In further embodiments, DMPatA or a pharmaceutically acceptable salt thereof, or des-methyl des-amino pateamine A (DMDAPatA) or a pharmaceutically acceptable salt thereof, is administered orally.

The present disclosure provides DMPatA or a pharmaceutically acceptable salt thereof, or des-methyl des-amino pateamine A (DMDAPatA) or a pharmaceutically acceptable salt thereof, for use in a method for treating pancreatic cancer.

In another aspect, the present disclosure provides a method for reducing the expression of C-MYC and Cyclin D1 in a subject, comprising administering to a subject in need thereof a therapeutically effective amount of des-methyl pateamine A (DMPatA) or a pharmaceutically acceptable salt thereof, or des-methyl des-amino pateamine A (DMDAPatA) or a pharmaceutically acceptable salt thereof.

In a further aspect, the present disclosure provides a method for decreasing hyaluronic acid and its receptor CD44 in the extracellular matrix of PDAC cells and tumors in a subject, comprising administering to a subject in need thereof a therapeutically effective amount of des-methyl pateamine A (DMPatA) or a pharmaceutically acceptable salt thereof, or des-methyl des-amino pateamine A (DMDAPatA) or a pharmaceutically acceptable salt thereof.

### DETAILED DESCRIPTION

Pancreatic ductal adenocarcinoma (PDAC) is one of the most refractory human malignancies; surgery is rarely curative, and better medical therapies are urgently needed. The eIF4F translation initiation complex regulates protein translation from a subset of mRNAs, frequently those involved in oncogenesis, including c-MYC. This complex initiates cap-dependent translation from a specialized 5′ modification on mRNA known as the 5′ cap.

In one aspect, the disclosure provides a method for treating pancreatic cancer. In one embodiment of the method, a therapeutically effective amount of des-methyl pateamine A (DMPatA), or a pharmaceutically acceptable salt thereof, is administered to a subject in need thereof. In another embodiment of the method, a therapeutically effective amount of des-methyl des-amino pateamine A (DMDAPatA), or a pharmaceutically acceptable salt thereof, is administered to a subject in need thereof. In certain embodiments of these methods, pancreatic cancer is pancreatic ductal adenocarcinoma.

Pharmaceutically acceptable salts may be formed from DMPatA or DMDAPatA and a pharmaceutically acceptable organic acid (e.g., carboxylic acid) or inorganic acid (e.g., mineral acid). Representative acids include hydrochloric acid, sulfuric acid, phosphoric acid, formic acid, acetic acid, trifluoroacetic acid, maleic acid, fumaric acid, succinic acid, tartaric acid, oxalic acid, citric acid, malic acid, benzoic acid, toluenesulfonic acid, methanesulfonic acid, and benzenesulfonic acid. Such salts may be formed during or after the synthesis of DMPatA or DMDAPatA.

In certain embodiments of the method, DMPatA or a pharmaceutically acceptable salt thereof, or des-methyl des-amino pateamine A (DMDAPatA) or a pharmaceutically acceptable salt thereof, is administered (dosed) intravenously, intraperitoneally, or orally.

In another aspect, the invention provides pharmaceutical compositions that include DMPatA or a pharmaceutically acceptable salt thereof, or des-methyl des-amino pateamine A (DMDAPatA) or a pharmaceutically acceptable salt thereof, and a pharmaceutically acceptable carrier for use in the treatment of pancreatic cancer (e.g., pancreatic ductal adenocarcinoma).

Suitable carriers include those suitable for administration to an animal (e.g., a human subject). Pharmaceutical compositions suitable for injectable use include sterile aqueous solutions (e.g., saline, dextrose) and dispersions.

In certain embodiments, DMPatA or a pharmaceutically acceptable salt thereof, or des-methyl des-amino pateamine A (DMDAPatA) or a pharmaceutically acceptable salt thereof, can be orally administered, for example, with an inert diluent or carrier, enclosed in hard or soft shell gelatin capsule, or compressed into tablets. For oral therapeutic administration, DMPatA or a pharmaceutically acceptable salt thereof, or des-methyl des-amino pateamine A (DMDAPatA) or a pharmaceutically acceptable salt thereof, can be combined with excipients and used in the form of ingestible tablets, buccal tables, troches, capsules, elixirs, suspensions, syrups, wafers, and the like. The amount of active compound in such therapeutically useful compositions is such that a suitable dosage is obtained.

In other embodiments, DMPatA or a pharmaceutically acceptable salt thereof, or des-methyl des-amino pateamine A (DMDAPatA) or a pharmaceutically acceptable salt thereof, can be administered parenterally or intraperitoneally. Solutions of the compounds as free base or pharmacologically acceptable salts can be prepared in water suitably mixed with additives, such as surfactants. Dispersions can also be prepared in oils.

In the methods, the term “therapeutically effective amount” refers to an amount effective, at dosages and for periods of time necessary, to achieve the desired therapeutic result, such as reduced levels of the protein products of MYC, CCND1, or CD44 genes. A therapeutically effective amount of a compound may vary according to factors such as the disease state, age, sex, and weight of the subject, and the ability of the compound to elicit a desired response in the subject. Dosage regimens can be adjusted to provide the optimum therapeutic response. A therapeutically effective amount is also one in which any toxic or detrimental effects of the administered compound are outweighed by the therapeutically beneficial effects.

It is to be noted that dosage values can vary with the severity of the condition to be alleviated. For any particular subject, specific dosage regimens can be adjusted over time according to the individual need and the professional judgment of the person administering or supervising the administration of the compositions. Dosage ranges are typically selected by a medical practitioner. The amount of active compound in the composition can vary according to factors such as the disease state, age, sex, and weight of the subject. Dosage regimens can be adjusted to provide the optimum therapeutic response. For example, a single bolus can be administered, several divided doses can be administered over time or the dose can be proportionally reduced or increased as indicated by the exigencies of the therapeutic situation.

In the methods, the administration of the compound can be systemic administration to the subject. The term “subject” includes mammalian organisms. Examples of subjects include humans and non-human mammals. In certain embodiments, the subject is a human.

The terms “administering,” “contacting,” or “treating” include any method of delivery of DMPatA, or a pharmaceutically acceptable salt thereof, or des-methyl des-amino pateamine A (DMDAPatA) or a pharmaceutically acceptable salt thereof, or a pharmaceutical composition comprising DMPatA or a pharmaceutically acceptable salt thereof, or des-methyl des-amino pateamine A (DMDAPatA) or a pharmaceutically acceptable salt thereof, into a subject's system.

In another aspect, the present disclosure provides a method for reducing the expression of C-MYC and Cyclin D1 in a subject, comprising administering to a subject in need thereof a therapeutically effective amount of des-methyl pateamine A (DMPatA) or a pharmaceutically acceptable salt thereof, or des-methyl des-amino pateamine A (DMDAPatA) or a pharmaceutically acceptable salt thereof.

In a further aspect, the present disclosure provides a method for decreasing hyaluronic acid and its receptor CD44 in the extracellular matrix of PDAC cells and tumors in a subject, comprising administering to a subject in need thereof a therapeutically effective amount of des-methyl pateamine A (DMPatA) or a pharmaceutically acceptable salt thereof, or des-methyl des-amino pateamine A (DMDAPatA) or a pharmaceutically acceptable salt thereof.

The present disclosure provides a pateamine A (PatA) derivative for targeting translation for the treatment of pancreatic ductal adenocarcinoma (PDAC). In one embodiment, the pateamine A derivative is des-methyl pateamine A (DMPatA) (also referred to herein a “MZ735” or “MZ”). In another embodiment, the pateamine A derivative is des-methyl des-amino pateamine A (DMDAPatA).

The chemical structures of pateamine A (PatA), DMDAPatA, and DMPatA are shown below.

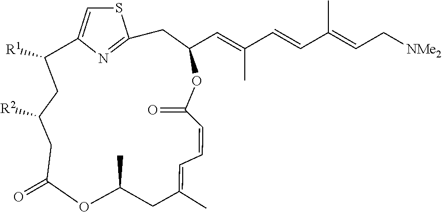

Pateamine A (PatA): R¹=Me, R²=NH₂
DMDAPatA: R¹, R²=H
DMPatA: R¹=H, R²=NH₂

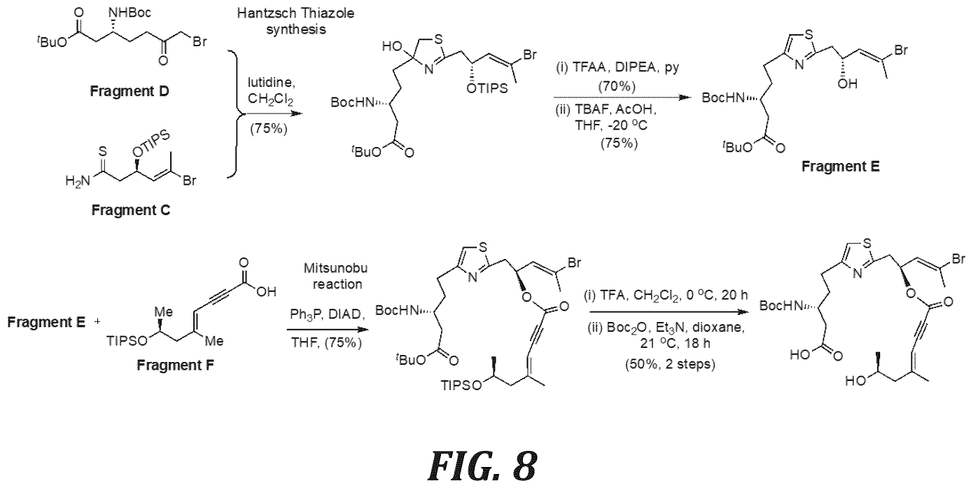

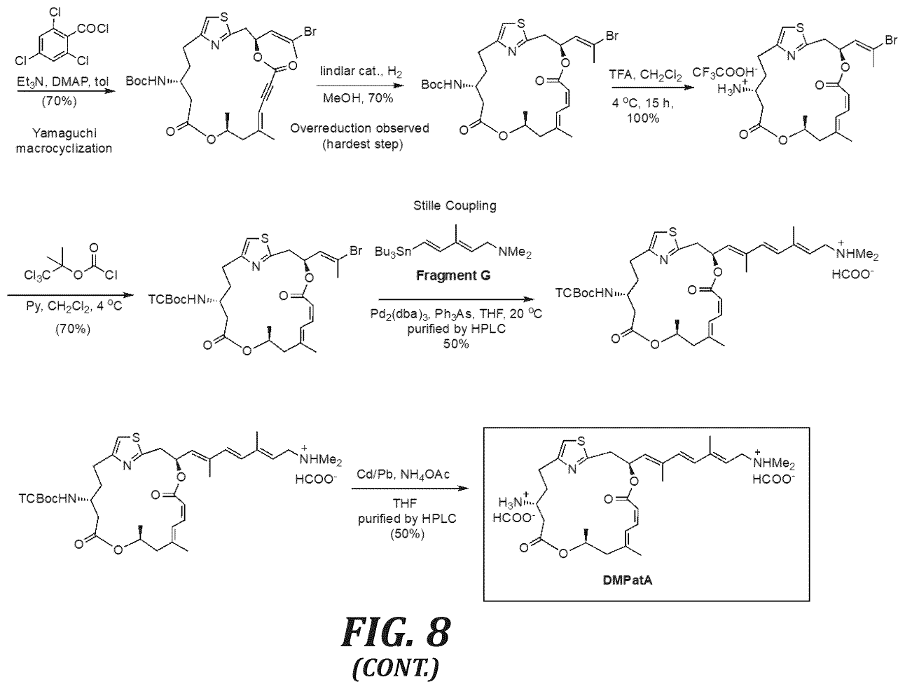

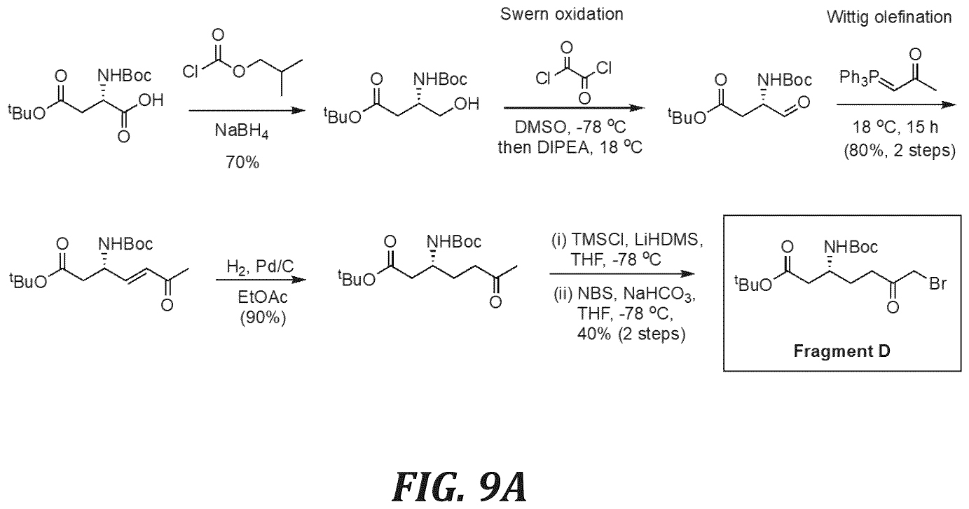

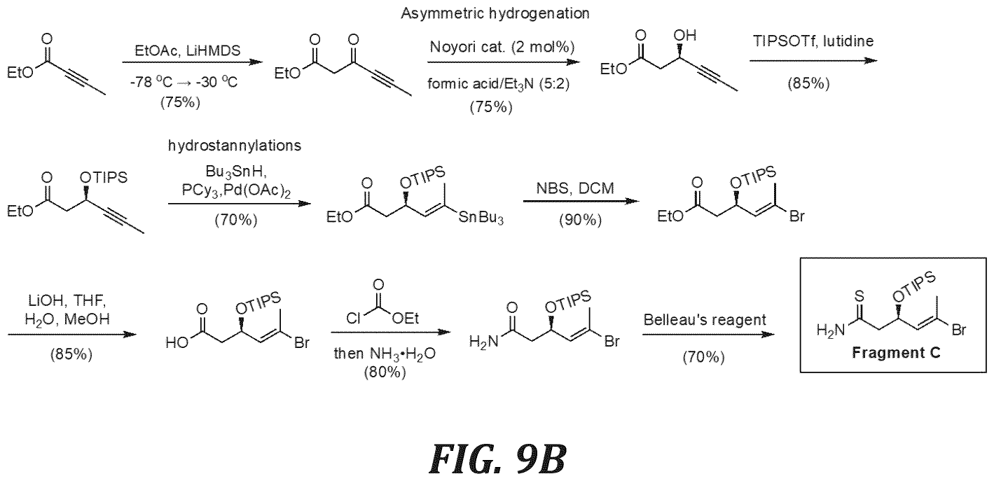

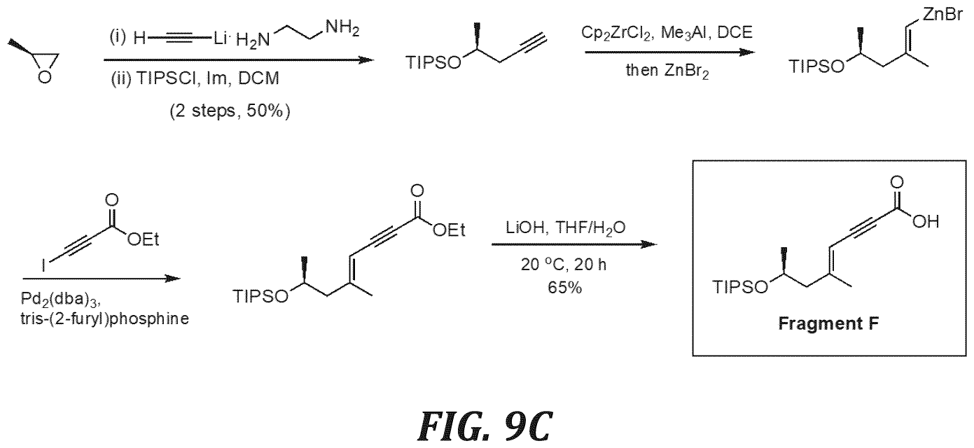

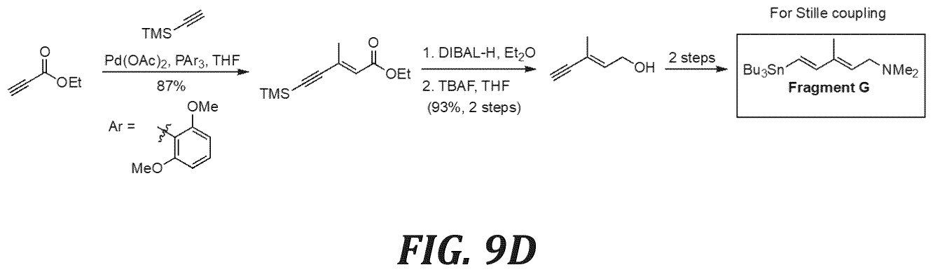

A synthesis of DMPatA is described in U.S. Pat. No. 10,889,596. A de novo synthesis of DMPatA is also described and illustrated schematically in FIG. 8. The de novo synthesis is efficient and scalable and is based on syntheses of fragments C, D, F and G as shown in FIGS. 9A-9D.

Data for DMDAPatA indicated that it is highly protein bound in human plasma and may lack sufficient in vivo potency required for clinical development. To address this, new PatA analogs were investigated with the goal of improving the physical properties and cytotoxic potency, which led to the discovery of DMPatA.

The present disclosure describes the efficacy of DMPatA and DMDAPatA in preferentially silencing relevant oncogene products in PDAC.

The present disclosure provides DMPatA and DMDAPatA as intraperitoneal therapeutics that are able to directly treat intraperitoneal carcinomatosis. As described herein, a safe intraperitoneal dose of 0.075-0.1 mg/kg in a mouse xenograph model has been demonstrated. This dose is able to reduce the volume of subcutaneously implanted tumors, showing that the intraperitoneal dose is sufficiently absorbed to provide therapeutic levels systemically.

The data described herein shows that targeting cap-dependent translation machinery with DMPatA and DMDAPatA, inhibitors of translation initiation factor 4A (eIF4A), influence both cellular and tumor microenvironment factors in PDAC cells. These PatA analogues prevent the proliferation of several PDAC cell lines and reduce the expression of important regulators of PDAC oncogenic behavior such as C-MYC and Cyclin D1. In addition, the data shows that a key micro-environmental factor, hyaluronic acid, which is known to be excessively deposited in the extracellular matrix of PDAC cells/tumors, and its receptor CD44, decrease following treatment with DMPatA and DMDAPatA.

One of the most difficult complications of pancreatic cancer is the development of peritoneal carcinomatosis, which may occur as the presenting manifestation of PDAC, much as occurs with ovarian cancer, or may appear late in the course of the disease. In either case, the prognosis for peritoneal carcinomatosis and cytology-positive ascites is grim, at times portending survival of only a few weeks. One major problem with carcinomatosis is that the peritoneal deposits are relatively avascular and do not respond well to systemic chemotherapy. The second major problem is that recurrent ascites requires frequent large volume paracentesis—which depletes the patient of protein and nutrients. As such, patients do not tolerate chemotherapy well and rapidly succumb to their disease.

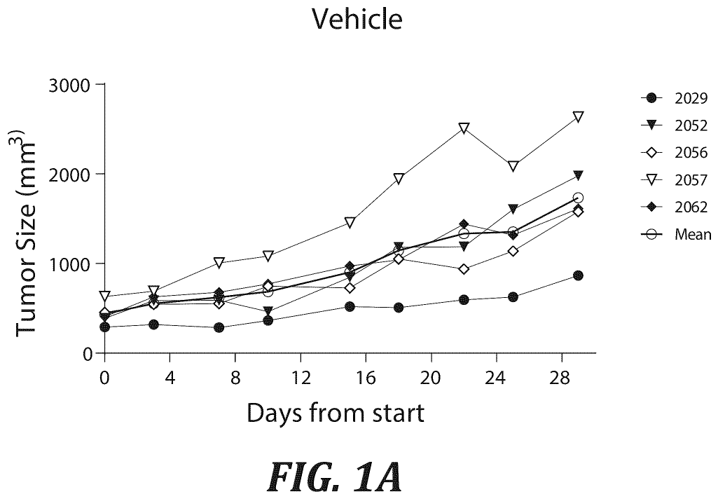

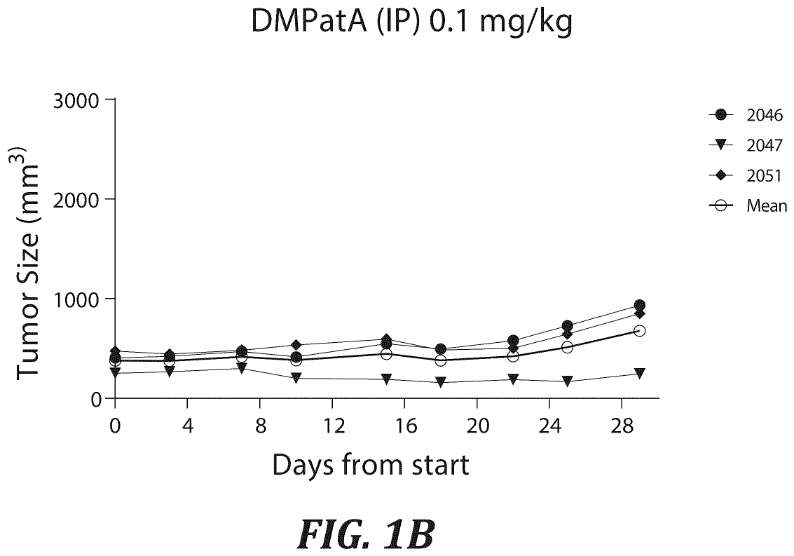

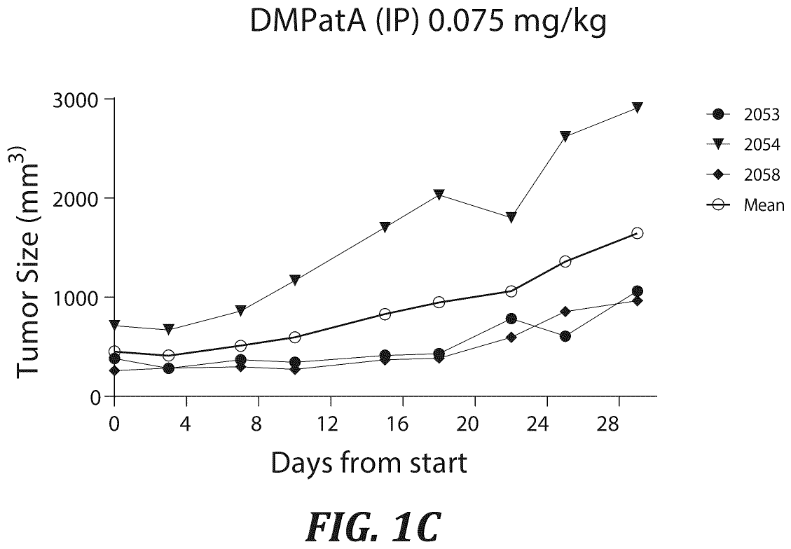

DMPatA and DMDAPatA are useful in an intraperitoneal therapy able to directly treat intraperitoneal carcinomatosis. A safe intraperitoneal (IP) dose of 0.075-0.1 mg/kg in a mouse xenograph model has been established. This dose is usually administered weekly. As such patients would likely require placement of an intraperitoneal catheter to allow intraperitoneal administration on a weekly or every other week basis. This dose is able to reduce the volume of subcutaneously implanted tumors, which demonstrates that DMPatA can be absorbed via the peritoneum. See Example 1. This dose is able to reduce the volume of subcutaneously implanted tumors. This data is presented in FIGS. 1A-1C.

FIG. 1A compares tumor size (mm³) growth over time (28 days) for the vehicle.

FIG. 1B compares tumor size (mm³) growth over time (28 days) for a dose of 0.1 mg/kg DMPatA.

FIG. 1C compares tumor size (mm³) growth over time (28 days) for a dose of 0.075 mg/kg DMPatA.

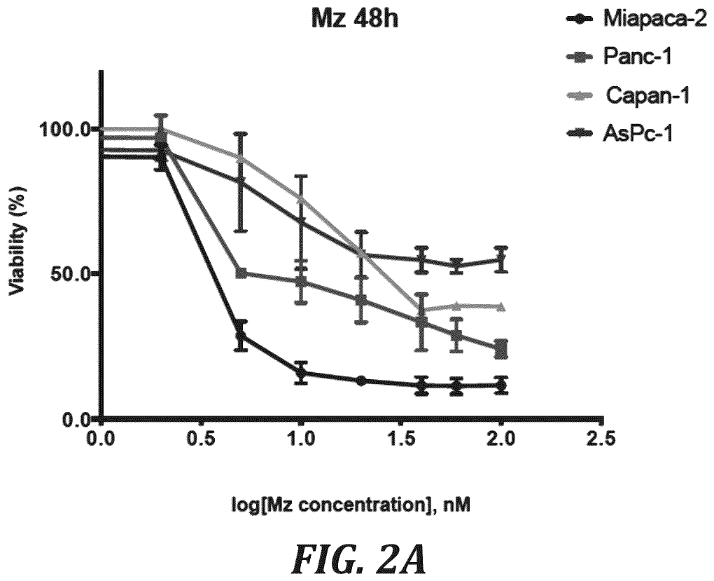

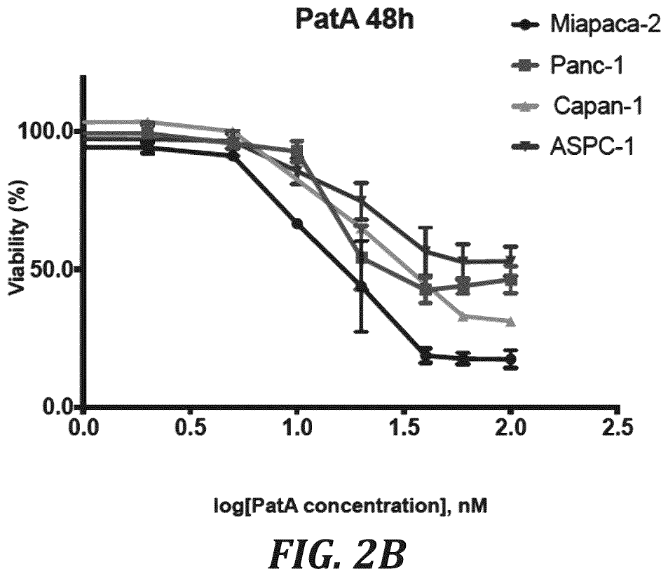

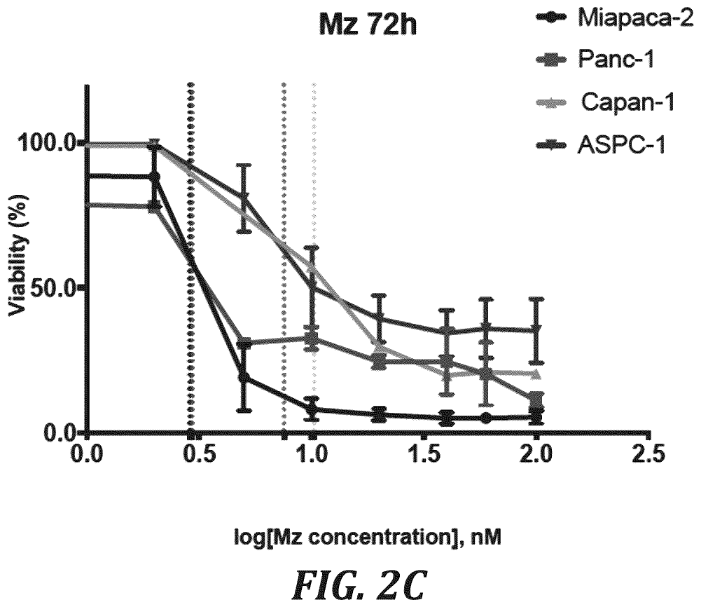

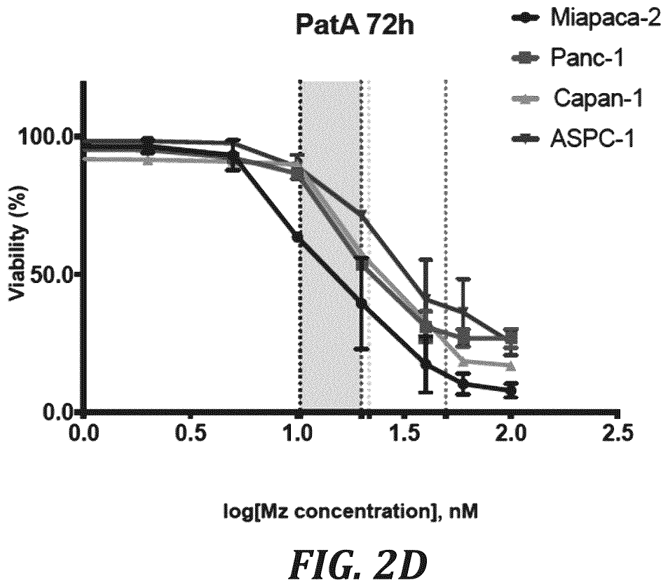

DMPatA and DMDAPatA are shown to inhibit cell proliferation in PDAC cells with Miapaca-2 being the most sensitive line. See Example 2. FIGS. 2A-2D are viability curves of DMDAPatA and DMPatA in four representative PDAC cell lines: MIA PaCa-2; PANC-1; Capan-1; and AsPC-1. DMPatA shows greater potency relative to DMDAPatA.

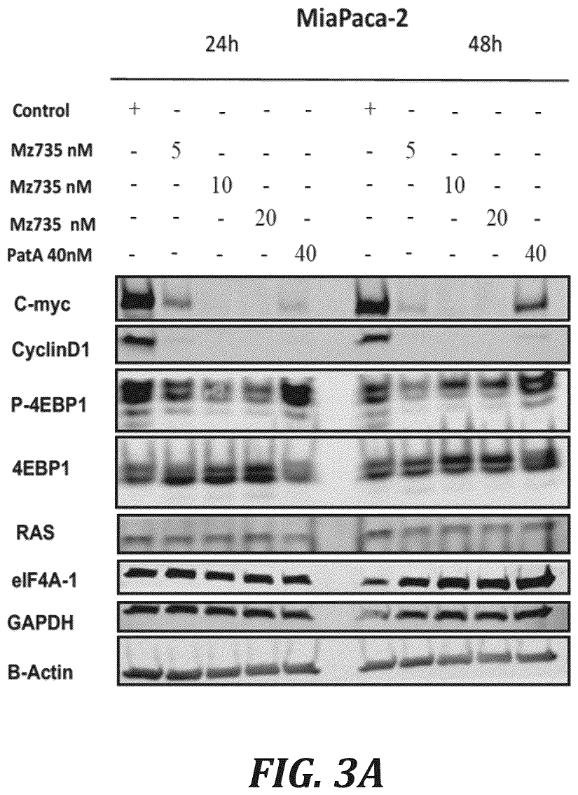

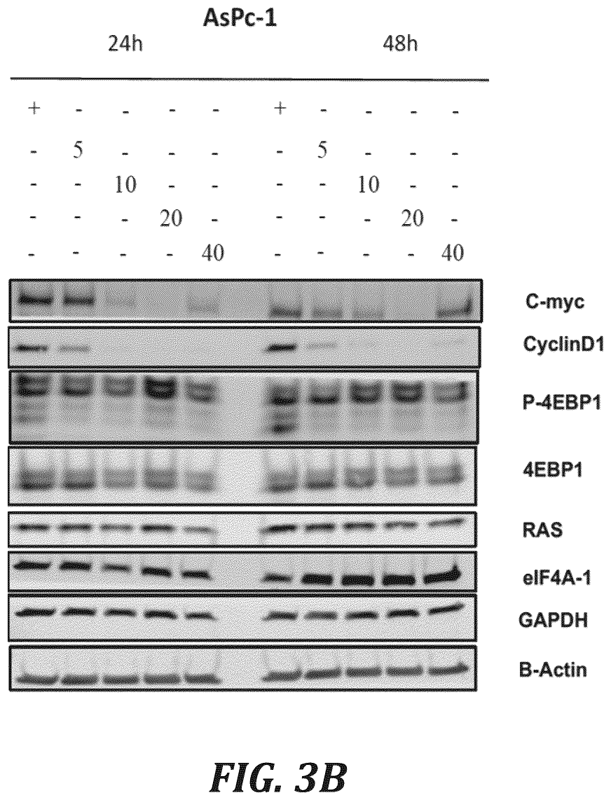

eIF4A inhibitors selectively repress the expression levels of key proteins in PDAC cell lines. FIGS. 3A and 3B are images of western blots showing the effect of DMDAPatA and DMPatA in PDAC cell lines (MIA PaCa-2 and AsPC-1). DMDAPatA and DMPatA markedly modify cellular factors by inhibiting the protein level of c-MYC and Cyclin D1 oncoproteins in PDAC cell lines (MIA PaCa-2 and AsPC-1). “Housekeeping proteins” GAPDH and beta-actin are stably expressed after DMPatA (MZ735). These proteins are commonly used as controls in western blot analysis and are so-called because they are typically not susceptible to agents that alter gene or protein expression.

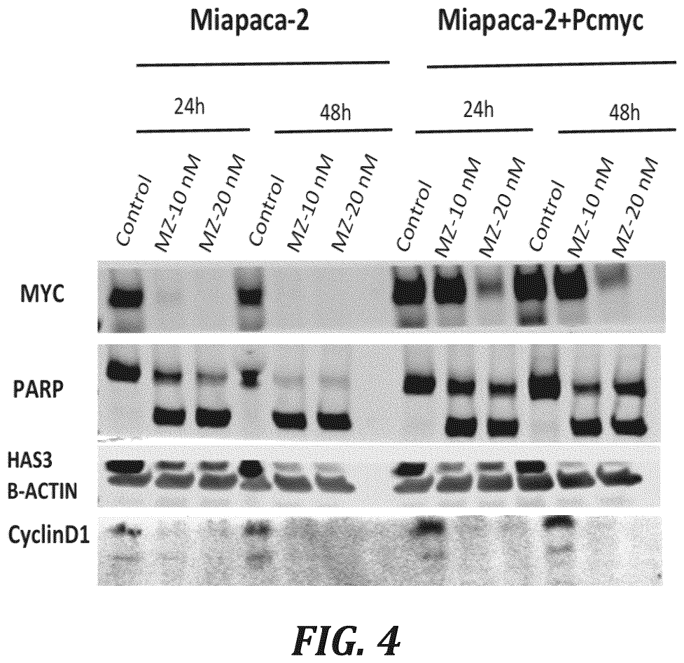

FIG. 4 compares images of western blots illustrating overexpression of MYC in MIA PaCa-3 transfected cells. The results show that overexpression of MYC did not rescue the observed drug effects on other proteins indicating the influence on these drugs on cap translation-dependent proteins rather than a consequence of MYC inhibition.

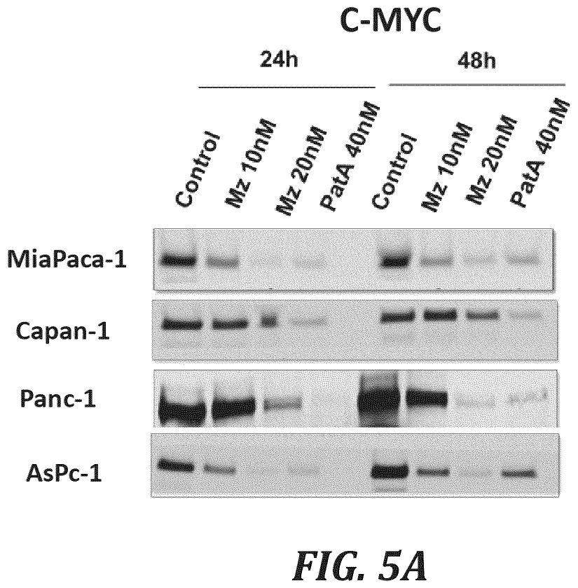

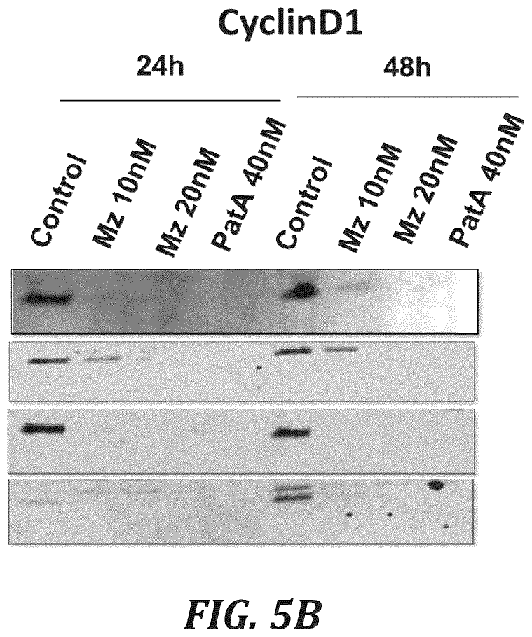

FIGS. 5A and 5B are images of western blots showing the effect of DMPatA and DMDAPatA on oncoproteins c-MYC and Cyclin D1 in PDAC cell lines: MIA PaCa-2; PANC-1; Capan-1; and AsPC-1.

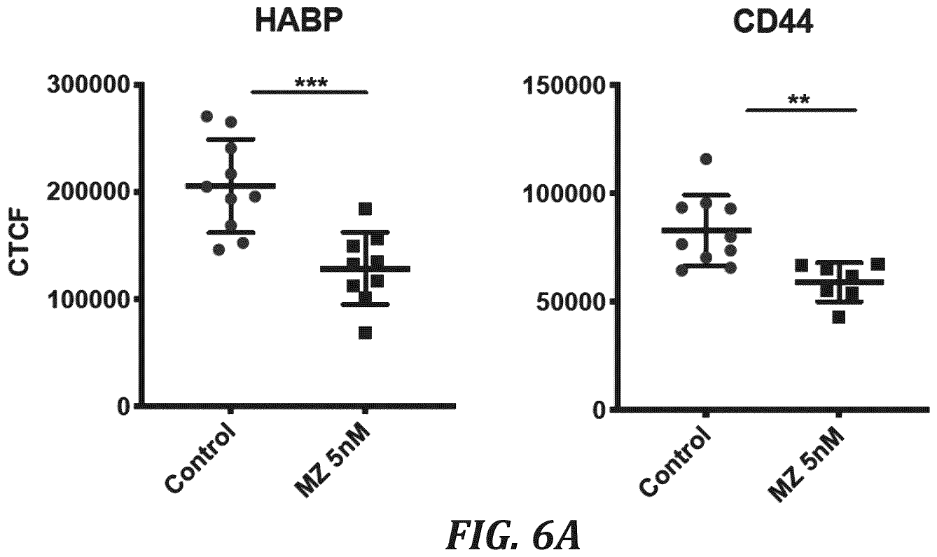

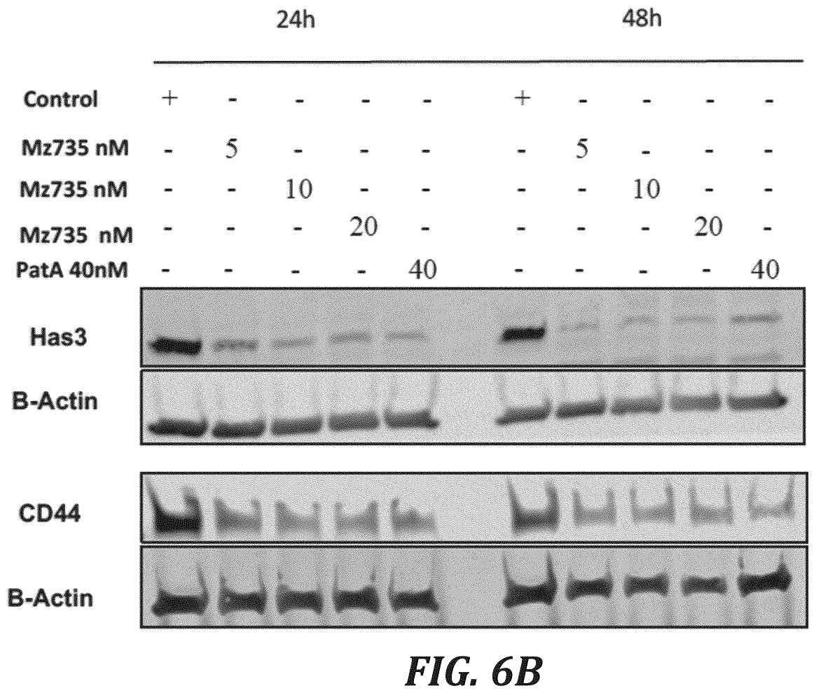

FIGS. 6A and 6B illustrate that representative eIF4A inhibitors (DMPatA and DMDAPatA) reduce the levels of hyaluronic acid and CD44. MIA PaCa-2 cells were treated with 5 nM of DMPatA for 24 h versus untreated control. Cells were stained for HA using biotinylated-HABP and anti-CD44. FIG. 6A compares the analysis of the corrected total cell fluorescence (CTCF) based on data extracted from confocal imaging (P<0.05) and FIG. 6B compares western blot showing the effect of DMPatA and DMDAPatA on stromal proteins HAS3 and CD44.

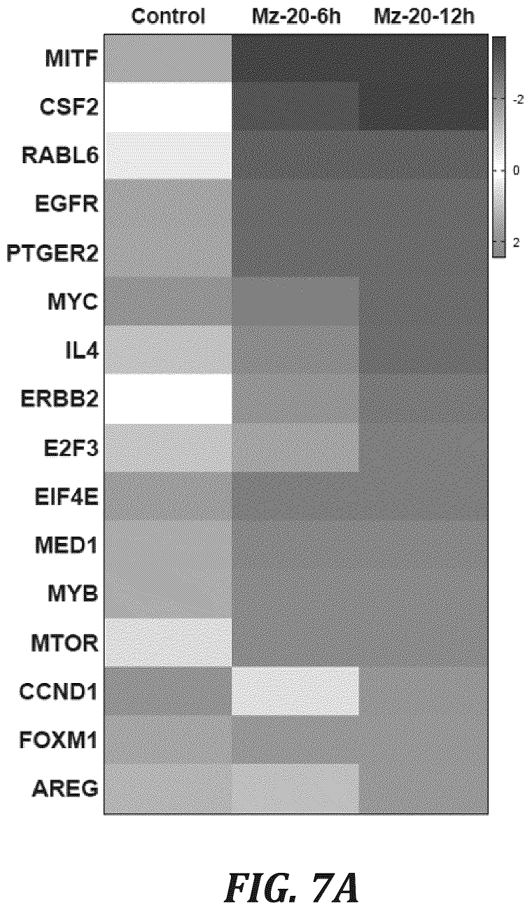

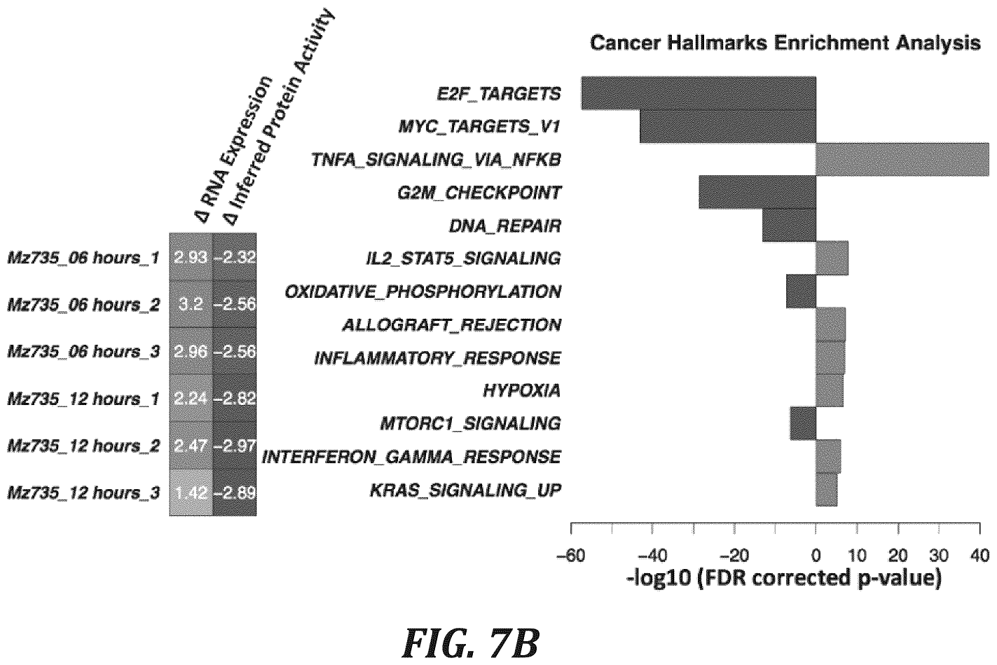

FIGS. 7A and 7B illustrate proteomics and RNA-seq analysis identify key regulatory molecules predicted to be responsible for the observed changes in the expression patterns following treatment with eIF4A inhibitors. FIG. 7A illustrates the IPA Upstream Regulator analysis used to identify key regulatory molecules predicted to be responsible for the differences observed in protein expression patterns following DMPatA treatment from a proteomics dataset. Proteins with Z-score>1 were considered to be activated (light gray bars) and those with Z-scores of <−1 were considered to be inhibited (dark gray bars). Heat map is showing only proteins with z-score cut off of ≤−1.8 for a total of 291 predicted upstream regulatory proteins. FIG. 7B illustrates the Virtual Inference of Protein-activity by Enriched Regulon analysis (VIPER) algorithm analysis used to identify differentially regulated master regulators, by examining data extracted from RNA-seq datasets of samples treated with DMPatA compared to untreated control. In the figures, MZ735 is DMPatA.

In a further aspect, the present disclosure provides a de novo synthesis of DMPatA. FIG. 8 is a schematic illustration of the de novo synthesis of DMPatA.

The de novo synthesis of DMPatA is efficient and scalable relying on the syntheses of fragments C, D, F and G. FIGS. 9A-9D are schematic illustrations of the syntheses of DMPatA Fragments D, C, F, and G, respectively.

The data presented herein show that targeting cap-dependent translation machinery with a translation initiation factor 4A (eIF4A) inhibitors DMPatA and DMDAPatA can influence both cellular and tumor microenvironment factors in PDAC cells. DMPatA and DMDAPatA prevent the proliferation of several PDAC cell lines and reduces the protein expression of important regulators of PDAC oncogenic behavior such as c-MYC and Cyclin D1. In addition, a key micro-environmental factor, hyaluronic acid, which is known to be excessively deposited in the extracellular matrix of PDAC cells/tumors, and its receptor CD44, have been shown to decrease following treatment with DMPatA and DMDAPatA.

### EXAMPLES

### Example 1

### Mouse Xenograph Model

In this example, the effectiveness of DMPatA in reducing tumor size in a mouse xenograph model is described.

Intraperitoneal therapy for PDAC. One of the most difficult complications of pancreatic cancer is the development of peritoneal carcinomatosis, which may occur as the presenting manifestation of PDAC, much as occurs with ovarian cancer, or may appear late in the course of the disease. In either case, the prognosis for peritoneal carcinomatosis and cytology-positive ascites is grim, at times portending survival of only a few weeks. One major problem with carcinomatosis is that the peritoneal deposits are relatively avascular and do not respond well to systemic chemotherapy. The second major problem is that recurrent ascites requires frequent large volume paracentesis—which depletes the patient of protein and nutrients. As such, patients do not tolerate chemotherapy well and rapidly succumb to their disease. Des-methyl pateamine A (DMPatA, MZ735) can be used as a novel intraperitoneal therapy able to directly treat intraperitoneal carcinomatosis. In certain embodiments, the safe intraperitoneal dose is 0.075-0.1 mg/kg, as established in a mouse xenograpgh model. This dose is able to reduce the volume of subcutaneously implanted tumors. The results are presented in FIGS. 1A-1C.

Fifteen female athymic nude mice were implanted with 5×10⁶ Mia-Paca-2 cells, injected subcutaneously into the right flank with 50% Matrigel. Once tumors reached 100-200 mm³ in volume, mice were randomized and enrolled into the study with three treatment groups including vehicle, MZ735 0.075, MZ735 0.1 mg/kg.

Mice were dosed once every four days interperitoneally (IP) for 28 days. Weights were taken twice per week to ensure overall health. All formulations were prepared on a day prior to, or on the day of dosing, using a serial dilution method and stored at 4° C. Formulations for this study were made using a vehicle of 10% Ethanol, 10% Cremophor EL, 4% glucose, and 76% PBS.

Body weights and tumor measurements were recorded twice weekly per OPTIC core-Tumor Growth Measurement at Columbia University.

At the end of the study mice were euthanized via CO₂ asphyxiation, per IACUC approved Euthanasia protocol at Columbia University.

### Example 2

### Cell Proliferation Assays

In this example, the effectiveness of DMPatA and DMDAPatA in reducing the proliferation of PDAC cancer cell lines is described. The results are presented in FIGS. 2A-2D.

Cell viability was measured using CellTiter-Glo® reagent (Promega, #G7570), according to the manufacturer's instructions. Briefly, cells were seeded at a density of 0.5×10⁶ cells in white 96-well plates and allowed to grow overnight. Cells were then treated with/without the combination and single agents as described earlier and incubated 37° C. and 5% CO₂ for appropriate time points. After incubation assay reagent was added to the cells and luminescent signal values were quantified using microplate reader (PHERAstar® FS, BMG LABTECH, Ortenberg, Germany).

While illustrative embodiments have been illustrated and described, it will be appreciated that various changes can be made therein without departing from the spirit and scope of the invention.

1. A method for treating pancreatic cancer, comprising administering to a subject in need thereof a therapeutically effective amount of des-methyl pateamine A (DMPatA) or a pharmaceutically acceptable salt thereof, or des-methyl des-amino pateamine A (DMDAPatA) or a pharmaceutically acceptable salt thereof.

2. The method of claim 1, wherein the pancreatic cancer is pancreatic ductal adenocarcinoma.

3. The method of claim 1, wherein DMPatA or a pharmaceutically acceptable salt thereof, or des-methyl des-amino pateamine A (DMDAPatA) or a pharmaceutically acceptable salt thereof, is administered intravenously, intraperitoneally, or orally.

4. The method of claim 1, wherein the subject is human.

5. A method for reducing the expression of C-MYC and Cyclin D1 in a subject, comprising administering to a subject in need thereof a therapeutically effective amount of des-methyl pateamine A (DMPatA) or a pharmaceutically acceptable salt thereof, or des-methyl des-amino pateamine A (DMDAPatA) or a pharmaceutically acceptable salt thereof.

6. The method of claim 5, wherein DMPatA or a pharmaceutically acceptable salt thereof, or des-methyl des-amino pateamine A (DMDAPatA) or a pharmaceutically acceptable salt thereof, is administered intravenously, intraperitoneally, or orally.

7. The method of claim 5, wherein the subject is human.

8. A method for decreasing hyaluronic acid and its receptor CD44 in the extracellular matrix of PDAC cells and tumors in a subject, comprising administering to a subject in need thereof a therapeutically effective amount of des-methyl pateamine A (DMPatA) or a pharmaceutically acceptable salt thereof, or des-methyl des-amino pateamine A (DMDAPatA) or a pharmaceutically acceptable salt thereof.

9. The method of claim 8, wherein DMPatA or a pharmaceutically acceptable salt thereof, or des-methyl des-amino pateamine A (DMDAPatA) or a pharmaceutically acceptable salt thereof, is administered intravenously, intraperitoneally, or orally.

10. The method of claim 8, wherein the subject is human.
